(19) United States
(12) Patent Application Publication
(10) Pub. No.: US 2022/0373486 A1
(43) Pub. Date: Nov. 24, 2022

(54) SCREENING/ANALYSIS OF FLUOROCARBONS USING X-RAY PHOTOELECTRON SPECTROSCOPY

(71) Applicant: The Penn State Research Foundation, University Park, PA (US)

(72) Inventors: Sara A. LINCOLN, State College, PA (US); Kalle L. JAHN, State College, PA (US); Jeffrey R. SHALLENBERGER, State College, PA (US); Demian M. SAFFER, State College, PA (US); Katherine H. FREEMAN, State College, PA (US)

(21) Appl. No.: 17/783,613

(22) PCT Filed: Jan. 11, 2021
(86) PCT No.: PCT/US21/12989

§ 371 Date: Jun. 8, 2022

Related U.S. Application Data
(60) Provisional application No. 62/958,952, filed on Jan. 9, 2020.

(51) Int. Cl. G01N 23/2273; G01N 1/40; G01N 33/18

(52) U.S. Cl. CPC G01N 23/2273; G01N 1/405; G01N 33/1826; G01N 2223/085; G01N 2223/637

(57) ABSTRACT

Methods of determining the presence or absence of fluorocarbon(s) on a substrate using X-ray Photoelectron Spectroscopy (XPS). A method may be used to determine the presence or absence of per- or polyfluoroalkyl substances PFASs. A method may use a porous polymer substrate. A method may use solid-phase extraction (SPE). A method may be used to determine the presence or absence of fluorocarbons in an aqueous sample. An aqueous sample may be a groundwater sample, wastewater sample, potable water sample, drinking water sample, or surface water sample. The limit of detection of fluorine in a method may be 0.05% F or less (for XPS analysis) and/or 20 ng or less on a substrate.

### CROSS REFERENCE TO RELATED APPLICATION

This application claims priority to U.S. Provisional Application No. 62/958,952, filed Jan. 9, 2020, the entire disclosure of which is incorporated herein by reference.

### BACKGROUND OF THE DISCLOSURE

Per- and polyfluoroalkyl substances (PFASs) are synthetic compounds that have been used in a wide range of consumer and industrial products since the mid-20^{th} century. Many of the more than 3000 PFASs that have been manufactured are present in the environment.

In firefighting, PFASs are effective in aqueous film-forming foams because they lower surface tension, enhancing the ability of films to spread and suppress flames. Firefighter training activities have released significant amounts of PFASs into the environment, sometimes leading to groundwater contamination; other sources of PFASs in the environment include consumer products, accidental industrial release, and wastewater treatment plants. PFASs have been documented in wildlife on a global scale since the 2000s, and perfluorooctanesulfonic acid (PFOS) is now listed under the Stockholm Convention on Persistent Organic Chemicals.

PFASs are receiving increased scientific and regulatory attention due to growing recognition of their health impacts and near ubiquity in the environment. A growing understanding of adverse health impacts associated with PFASs has driven the United States Environmental Protection Agency (EPA) to issue a drinking water Lifetime Health Advisory (LHA) of 70 ng/L for PFOS and perfluorooctanoic acid (PFOA), two of the most widely detected and studied PFASs. The Third Unregulated Drinking Water Contaminants list includes PFOA, PFOS, and four additional PFASs, and EPA method 537.1 was extended to include protocols for measuring 18 PFASs in 2018. Commercial laboratories now offer analysis of more than 30 PFASs.

While the list of measurable PFAS analytes is expanding, comprehensive detection of the full suite of structures is elusive. PFAS assemblages in the environment—and in humans—are complex and the dominant species released into the environment have changed over time. Changes in PFAS manufacturing are partly responsible: early synthetic processes used electrochemical fluorination, which creates a mixture of approximately 70% straight-chained and 30% branched-chained isomers of perfluoroalkyl acids, while the “cleaner” telomerization process developed in the 1970s creates primarily straight-chained PFAS. PFOS, for instance, has commonly been detected in the environment as a mixture of 10 branched isomers and the linear isomer. Manufacturers have voluntarily phased out PFOS and PFOA in the United States due to environmental and toxicological concerns, but production continues in some countries and release from legacy products is a continuing risk. Additionally, when a PFAS is phased out, it is typically replaced by a structurally similar compound for which less toxicological data may be available, increasing the number of PFASs that have been mass-produced, and contributing to an ever-changing global PFAS landscape.

Comprehensive detection of PFASs in groundwater is challenging because methods commonly used to measure PFASs at environmentally relevant trace concentrations target a limited suite of PFASs, and the structural diversity of the compound class prevents total PFAS measurement in a single analytical window using mass spectrometry. Analytical issues pose problems for comprehensive measurement of PFASs by mass spectrometry. The high structural diversity of PFASs precludes their measurement in a single analytical window. Liquid chromatography with tandem mass spectrometry (LC/MS/MS) is well suited for the analysis of ionic species such as PFOS, whereas neutral and nonionic PFASs including fluorotelomer alcohols, perfluoroalkane sulfonamides, and perfluoroalkane sulfonamido ethanols may be better detected using gas chromatography/tandem mass spectrometry (GC/MS/MS; e.g., ITRC 2018). LC/ and GC/MS/MS approaches in common use rely on targeted analysis (selected ion and multiple reaction monitoring) for detection of PFAS at the trace concentrations at which they are typically present in the environment; as a result, they can only detect known compounds. Less widely available high- and ultra-high-resolution mass spectrometry can be used for more exploratory, untargeted analysis, but the limited number of authentic reference standards available hinders confirmation of the identity of novel compound classes such as those used in “replacement chemistries”; fewer than 100 of the more than 3000 known PFASs are currently available commercially as analytical standards.

The Total Oxidizable Precursor assay (TOP) is the most inclusive method for detecting PFASs in widespread use. Through a six-hour oxidation step in which samples are exposed to hydroxyl radicals, the TOP assay converts known and unknown PFAS precursors to stable PFASs that can be measured using LC/MS/MS. Any PFAS precursors that are not susceptible to oxidation by hydroxyl radicals remain intact and evade detection, and the limitations of targeted detection described previously also apply to the TOP assay.

Recognition that LC/MS/MS methods targeting specific PFASs can lead to underdetection of complex mixtures of PFASs in environmental samples has fueled interest in more comprehensive detection methods. Existing techniques for bulk organofluorine measurement, however, have not been widely adopted because their relatively high limits of detection are inadequate for trace environmental detection of PFASs. ¹⁹F NMR and combustion ion chromatography following preparatory chemistry to isolate organofluorine-containing fractions both require PFAS concentrations in the μg/L range. Particle-Induced Gamma Ray Emission (PIGE) spectroscopy, a sensitive surface analysis technique that can detect total F, has been used to measure the PFAS content of papers and textiles but it cannot differentiate between inorganic and organic F. PIGE has been used to measure total F in dried water residues but the high fluoride content of most natural waters is likely to overwhelm any PFAS signal.

Based on the foregoing, there exists an ongoing and unmet need for improved methods of PFAS analysis.

### SUMMARY OF THE DISCLOSURE

This disclosure provides novel methods for screening/analyzing samples, such as, for example, groundwater for total PFASs using XPS. In various examples, a method for detecting one or more fluorocarbon(s) in a sample (which may be an aqueous sample) suspected of having one or more fluorocarbon(s), where the sample (or an isolated and/or concentrated sample) is disposed on at least a portion of a substrate, comprises: subjecting the sample to x-ray on the substrate (e.g., in the sample). The presence of fluorine in the sample as determined by the XPS analysis is indicative of the presence or absence of fluorocarbon(s) on the substrate (e.g., in the sample).

XPS analysis may comprise determining the strength and/or position of the F 1s peak in the spectrum. This determination may be carried out by using high-resolution scans. The presence or absence of various fluorocarbons on a substrate can be determined. The presence or absence of various combinations of fluorocarbons on a substrate may be determined.

Various substrates can be used in the methods. A substrate may be a porous polymer membrane, which may be a non-fluorinated porous polymer membrane. It may be desirable that the porous polymer membrane is a non-fluorinated porous polymer membrane. In various non-limiting examples, a non-fluorinated porous polymer membrane has a nominal pore size (e.g., an average pore size) of about 0.001 to about 1 micron, including all 0.001 micron values and ranges therebetween. In various other non-limiting examples, a non-fluorinated porous polymer membrane has a nominal pore size (e.g., an average pore size) of 0.2 to 0.8 microns or 0.3 to 0.6 microns.

A sample may be isolated and/or concentrated. A sample may be prepared using a polymeric adsorption media. The polymeric adsorption media may be housed in a solid phase extraction (SPE) cartridge. In a non-limiting example, the substrate is prepared using solid-phase extraction (SPE) according to a standard method (e.g., an EPA method such as, for example, EPA method 537, and the like).

A substrate for use in a method may be prepared using various samples suspected of having one or more fluorocarbon(s). A sample that is subjected to XPS analysis may be derived from another sample. For example, a sample is an aqueous sample or is derived (e.g., isolated and/or concentrated) from an aqueous sample.

The XPS analysis may comprise irradiating a region of the substrate with x-rays, and detecting emitted photoelectrons with a spectrometer. The incident x-rays may have various angles with respect to a surface of the substrate. It may be desirable that the incident x-rays are at an angle of less than 90 degrees and greater than 0 degrees with respect to a surface (or at least a portion of a surface) of the substrate (e.g., a 45 degree angle) of the region of the substrate.

A method may comprise quantifying the amount of fluorine (e.g., organofluorocarbon(s)) in the sample. In non-limiting examples, the amount of fluorine in the sample correlates to the amount of fluorocarbon(s) in the sample or the sample on the substrate. The fluorine quantification may be carried out by comparison to (e.g., correlation to) one or more standards (e.g., standard sample(s) and the like).

### BRIEF DESCRIPTION OF THE FIGURES

For a fuller understanding of the nature and objects of the disclosure, reference should be made to the following detailed description taken in conjunction with the accompanying figures.

FIG. 1 shows standard curve relating perfluoroalkyl moiety concentration and XPS % fluorine measured on PFOS standards. A linear regression (% F=0.0085×moiety concentration−0.2044, R²=0.878) is plotted with a 95% confidence interval.

FIG. 2 shows comparison of perfluoroalkyl moiety concentrations in two groundwater samples (Well A and Well B) measured using different techniques: PFOS and PFOA only by LC/MS/MS (light gray), 32 PFAS by LC/MS/MS after oxidizable precursor breakdown by TOP assay (medium gray), and XPS (dark gray) (left to right in each group). Error bars on XPS results represent variability introduced by the standard error on the linear regression coefficients used to convert % F to perfluoroalkyl moiety concentration. The EPA Health Advisory level of 70 ng/L combined PFOS/PFOA was converted to a perfluoroalkyl moiety range calculated by assuming the 70 ng/L consisted of either 100% PFOS or 100% PFOA.

FIG. 3 shows PFAS analytical results for groundwater samples (providing Table 2).

### DETAILED DESCRIPTION OF THE DISCLOSURE

Although claimed subject matter will be described in terms of certain embodiments/examples, other embodiments/examples, including embodiments/examples that do not provide all of the benefits and features set forth herein, are also within the scope of this disclosure. Various structural, logical, and process step changes may be made without departing from the scope of the disclosure.

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all steps and compositions of matter described herein in the text and figures of this disclosure, including all such steps individually and in all combinations thereof.

Throughout this application, the singular form encompasses the plural and vice versa. All sections of this application, including any supplementary sections or figures, are fully a part of this application.

This disclosure provides methods for detecting fluorocarbons in a sample using X-ray Photoelectron Spectroscopy (XPS). This disclosure includes all steps of the methods described herein. Thus, in an example, a method consists essentially of a combination of the steps of the methods disclosed herein. In another example, a method consists of such steps.

XPS is a surface-sensitive spectroscopic technique capable of detecting all elements with the exception of H and He. XPS can also determine the chemical state(s) of elements in many cases, and may distinguish fluorocarbons from inorganic F. The strong C—F bond, largely responsible for the persistence of PFASs in the environment, creates a distinct signal that makes fluorocarbons readily detectable by XPS. XPS has not, to our knowledge, been used to measure PFASs in environmental samples. For example, in the present disclosure methods for concentrating PFASs from water using solid phase extraction (SPE) were combined with XPS, in novel methods for measuring total fluorocarbons in groundwater samples.

This disclosure provides novel methods for screening/analyzing, for example, groundwater for total PFASs using XPS. In an embodiment, this method consistently detects PFASs at concentrations ≥25 ng/l, and the linear correlation between the perfluoroalkyl moiety concentration and % F enables semi-quantification. In another embodiment, the disclosed methods can be used to measure groundwater samples from former fire training sites.

For comparative purposes, PFASs, including those released from precursors by chemical oxidation, were measured using XPS and LC/MS/MS. In an embodiment, the methods described herein using XPS reveal higher concentrations of fluorocarbons, suggesting the presence of a more complex mixture of PFASs not fully captured by conventional mass spectrometry techniques. Overall, this XPS method may be used as a screening/analytical tool for identifying PFAS-contaminated water samples.

It was surprisingly found that methods of the present disclosure provide unexpected limits of detection for fluorocarbon(s) in a sample (e.g., a sample disposed on a substrate). For example, a limit of detection (LOD) was 0.05% or less F was achieved using methods of the present disclosure. In another example, a limit of detection (LOD) for fluorocarbon(s) on a substrate was 20 nm or less was achieved using methods of the present disclosure.

In various examples, a method for detecting one or more fluorocarbon(s) in a sample (which may be an aqueous sample) suspected of having one or more fluorocarbon(s), where the sample (or an isolated and/or concentrated sample) is disposed on at least a portion of a substrate, comprises: subjecting the sample to x-ray on the substrate (e.g., in the sample). The presence of fluorine in the sample as determined by the XPS analysis is indicative of the presence or absence of fluorocarbon(s) on the substrate (e.g., in the sample).

The XPS analysis may comprise determining the strength and/or position of the F 1s peak in the spectrum. This determination may be carried out by using high-resolution scans. The F is peak position can be used to distinguish fluorocarbons (e.g., PFAS and related fluorocarbons (687.0-689.5 eV) from inorganic fluorides (684.0-686.0 eV).

The presence or absence of various fluorocarbons on a substrate can be determined. The presence or absence of various combinations of fluorocarbons on a substrate may be determined. A fluorocarbon comprises one or more fluorine atoms(s), where each of the fluorine atom(s) is/are covalently bound to a carbon. The fluorocarbon may be a polyfluorinated fluorocarbon and/or a perfluorinated fluorocarbon.

Non-limiting examples of fluorocarbons include fluoroalkyl compounds(s) (e.g., perfluorooctanoic acid (PFOA), perfluorooctanesulfonic acid/perfluorooctanesulfonate (PFOS), perfluorobutane sulfonic acid (PFBS), hexafluoropropylene oxide, and the like, and combinations thereof), and the like, and combinations thereof.

Various substrates can be used in the methods. The substrate may be a porous polymer membrane, which may be a non-fluorinated porous polymer membrane. In an example, the non-fluorinated porous polymer membrane does not have any detectible fluorine (e.g., detectible by XPS analysis). Non-fluorinated porous polymer membranes may comprise various polymers. Non-limiting examples of polymers include hydrocarbon polymers (e.g., polyethylenes, polypropylenes, and the like), polyethersulfones (PESs), polyesters (e.g., nylons and the like), cellulose materials (e.g., cellulose acetate and the like), polycarbonates, functionalized analogs thereof, and the like, and combinations thereof. Non-fluorinated porous polymer membranes may have various porosity. In various non-limiting examples, a non-fluorinated porous polymer membrane has a nominal pore size (e.g., an average pore size) of about 0.001 to about 1 micron, including all 0.001 micron values and ranges therebetween. In various other non-limiting examples, a non-fluorinated porous polymer membrane has a nominal pore size (e.g., an average pore size) of 0.8 to 0.2 microns or 0.6 to 0.3 microns. Non-limiting examples of porous polymer membranes include porous cellulose acetate membranes, which may have a pore size of about 0.45 microns. However, use of other porous polymer membranes and other pore sizes are within the scope of this disclosure. A sample may be isolated and/or concentrated. A sample may be prepared using a polymeric adsorption media. The polymeric adsorption media may be housed in a solid phase extraction (SPE) cartridge. For example, the SPE cartridge media is styrene-divinylbenzene (SDVB) polymer modified with a nonpolar surface. In a non-limiting example, Agilent Bond-Elut PPL, 1 g, 6 ml cartridges are used. However, use of other SPE cartridges is within the scope of this disclosure.

In a non-limiting example, the substrate is prepared using solid-phase extraction (SPE). The solid-phase extraction may comprise contacting an aqueous sample with an SPE cartridge comprising a polymeric adsorption media (if fluorocarbon(s) are present in the aqueous sample, at least a portion of the fluorocarbon(s) are disposed on the SPE cartridge polymeric adsorption media), eluting at least a portion of the fluorocarbon(s), if present, from the SPE cartridge polymeric adsorption media to form an SPE eluent, and optionally, concentrating the SPE eluent to form a concentrated eluent (e.g., by heating the SPE eluent in an inert atmosphere). The eluent or the concentrated eluent may be contacted with the substrate or a portion thereof and at least a portion of the fluorocarbon(s) are disposed on the substrate, which may be used directly (e.g., without alteration after preparation) in the XPS analysis. In a non-limiting example, the substrate is prepared using solid-phase extraction (SPE) according to a standard method (e.g., an EPA method such as, for example, EPA method 537, and the like).

A substrate for use in a method may be prepared using various samples suspected of having one or more fluorocarbon(s). A sample that is subjected to XPS analysis may be derived from another sample. For example, a sample is an aqueous sample or is derived (e.g., isolated and/or concentrated) from an aqueous sample. Non-limiting examples of samples suspected of having one or more fluorocarbon(s) include aqueous samples, such as, for example, groundwater samples, wastewater samples, potable water samples, drinking water samples, surface water samples, and the like. Non-limiting examples of wastewater samples include private wastewater samples, municipal wastewater samples, industrial wastewater samples, and the like.

The XPS analysis may comprise irradiating a region of the substrate with x-rays, and detecting emitted photoelectrons with a spectrometer. The incident x-rays may have various angles with respect to a surface of the substrate. It may be desirable that the incident x-rays are at an angle of less than 90 degrees and greater than 0 degrees with respect to a surface (or at least a portion of a surface) of the substrate (e.g., a 45 degree angle) of the region of the substrate.

Various x-ray photoelectron spectrometers may be used the methods. In a non-limiting example, a Physical Electronics VersaProbe II instrument equipped with a monochromatic Al kα x-ray source (hν=1,486.7 eV) and a concentric hemispherical analyzer. However, use of other x-ray photoelectron spectrometers is within the scope of this disclosure.

A method may comprise quantifying the amount of fluorine (e.g., organofluorocarbon(s)) in the sample. In non-limiting examples, the amount of fluorine in the sample correlates to the amount of fluorocarbon(s) in the sample or the sample on the substrate. The fluorine quantification may be carried out by comparison to (e.g., correlation to) one or more standards (e.g., standard sample(s) and the like). For example, fluorine quantification is carried out by comparison of the x-ray photoelectron fluorine signal to a standard curve that is based on samples having known fluorine content.

The steps of the methods described in the various embodiments and examples disclosed herein are sufficient to carry out the methods of the present disclosure. Thus, in an example, a method consists essentially of a combination of the steps of the methods disclosed herein. In various other examples, a method consists of such steps.

The following Statements describe various examples of methods of the present disclosure:

Statement 1. A method for detecting one or more fluorocarbon(s) (e.g., per- and polyfluoroalkyl substances, PFAS(s)) in a sample suspected of having one or more fluorocarbon(s), where the sample is disposed on at least a portion of a substrate, comprising: subjecting the sample to x-ray photoelectron spectroscopy (XPS) analysis (e.g., obtaining an XPS spectrum of the sample determine if fluorine is present on the sample), where the presence of a fluorine signal as determined by XPS analysis is indicative of the presence of fluorine (e.g., one or more fluorocarbon(s)) in the sample or the absence of a fluorine signal as determined by XPS analysis is indicative of the absence of fluorine (e.g., one or more fluorocarbon(s)) in the sample.
Statement 2. The method according to Statement 1, where the XPS analysis comprises determining the strength and/or position of the F 1s peak.
Statement 3. The method according to Statement 1 or Statement 2, where the XPS analysis comprises one or more high-resolution scans. The position of the F is peak, which may be determined by high-resolution XPS scans, may distinguish fluorocarbons (687.0-689.5 eV) from inorganic fluorides (684.0-686.0 eV)).
Statement 4. The method according to any one of the preceding Statements, where the fluorocarbon compound(s) is/are chosen from fluoroalkyl compounds(s) (e.g., perfluorooctanoic acid (PFOA), perfluorooctanesulfonic acid/perfluorooctanesulfonate (PFOS), perfluorobutane sulfonic acid (PFBS), hexafluoropropylene oxide, and the like, and combinations thereof), and the like, and combinations thereof.
Statement 5. The method according to any one of the preceding Statements, where the substrate comprises a porous polymer membrane.
Statement 6. The method according to Statement 5, where the porous polymer membrane is chosen from hydrocarbon polymers (e.g., polyethylenes, polypropylenes, and the like), polyethersulfones (PESs), polyesters (e.g., nylons and the like), cellulose materials (e.g., cellulose acetate and the like), polycarbonates, functionalized analogs thereof, and the like, and combinations thereof.
Statement 7. The method according to any one of the preceding Statements, where the sample is an aqueous sample and/or the method further comprising preparing the sample using an aqueous sample. The preparing may include isolation and/or concentration of the sample.
Statement 8. The method according to Statement 7, where the aqueous sample is chosen from groundwater samples, wastewater samples (e.g., private wastewater samples, municipal wastewater samples, industrial wastewater samples, and the like), potable water samples, drinking water samples, surface water samples, and the like.
Statement 9. The method according to Statement 7 or Statement 8, where the preparing comprises using solid-phase extraction (SPE).
Statement 10. The method according to Statement 9, where the using solid-phase extraction comprises contacting the aqueous sample with an SPE cartridge comprising a polymeric adsorption media, where, if fluorocarbon(s) are present in the aqueous sample, at least a portion of the fluorocarbon(s) are disposed on the SPE cartridge polymeric adsorption media, eluting at least a portion of the fluorocarbon(s), if present, from the SPE cartridge polymeric adsorption media to form an SPE eluent, and optionally, concentrating the SPE eluent to form a concentrated eluent (e.g., by heating the SPE eluent in an inert atmosphere).
Statement 11. The method according to Statement 10, where the eluent or the concentrated eluent is contacted with the substrate or a portion thereof and at least a portion of the fluorocarbon(s) are disposed on the substrate, which may be used directly (e.g., without alteration after preparation) in the XPS analysis.
Statement 12. The method according to any one of the preceding Statements, where the XPS analysis comprises irradiating a region of the substrate with x-rays, and detecting emitted photoelectrons with a spectrometer. In a non-limiting example, the incident x-rays are at an angle of less than 90 degrees and greater than 0 degrees with respect to a surface (or at least a portion of a surface) of the substrate (e.g., a 45 degree angle) of the region of the substrate.
Statement 13. The method according to any one of the preceding Statements, further comprising quantifying the amount of fluorine in the sample, where the amount of fluorine in the sample correlates to the amount of fluorocarbon(s) in the sample.
Statement 14. The method according to Statement 13, where the fluorine quantification is carried out by comparison to (e.g., correlation to) one or more standard sample(s) (e.g., fluorine quantification is carried out by comparison of the x-ray photoelectron fluorine signal to a standard curve that is based on samples having known fluorine content).
Statement 15. The method according to any one of the preceding Statements, where the limit of detection of fluorine in the sample is 0.05% F or less (for XPS analysis) and/or 20 ng or less on a substrate (e.g., for PFAS on a substrate).

The following example(s) is/are provided to illustrate the present disclosure, and are not intended to be limiting.

### Example 1

This example describes the detection of total per- and polyfluoroalkyl substances (PFAS) in groundwater using X-ray photoelectron spectroscopy.

Materials and Methods

Groundwater Sampling. Water samples were collected from two monitoring wells (Wells A and B) at a former firefighter training site in University Park, Pa. The site was historically associated with the release of chlorinated volatile organic compounds (CVOCs), specifically tetrachloroethene (PCE) and trichloroethene (TCE). Groundwater remediation for CVOCs at the site began in 2001; PFASs were identified as additional contaminants in 2015. Groundwater from three wells was pumped into a treatment system that removed CVOCs with a perforated plate air stripper and PFASs with granulated activated carbon (GAC). A shallow vadose zone infiltration gallery returned treated water to the subsurface.

Solid phase extraction and sample preparation. PFAS were extracted from one-liter water samples following the solid phase extraction (SPE) steps outlined in EPA Method 537. Briefly, SPE cartridges (Agilent Bond-Elut PPL, 1 g, 6 ml cartridges) were pre-conditioned with 20 ml of methanol (Optima grade, Fisher Chemical) and 20 ml of MilliQ water (previously found to have no detectable PFAS), followed by sample water transferred through low density polyethylene tubing at a vacuum pressure of 15-20 mm Hg. After extraction, the cartridges were dried with air and PFASs were eluted with 8 ml of methanol that was first used to rinse sample bottles.

The methanol extracts were concentrated to volumes of ˜0.5 ml under N₂ in a water bath (50° C.) and transferred to polyethylene microcentrifuge tubes. Extracts were further concentrated under N₂ on a heating block (50° C.) to a volume of 3-5 μl. The concentrated extracts were transferred to 3.175 mm diameter cellulose acetate membrane filters 1 μl at a time using a pipet; filters were allowed to dry 5 min after each 1 μl transfer of the extract.

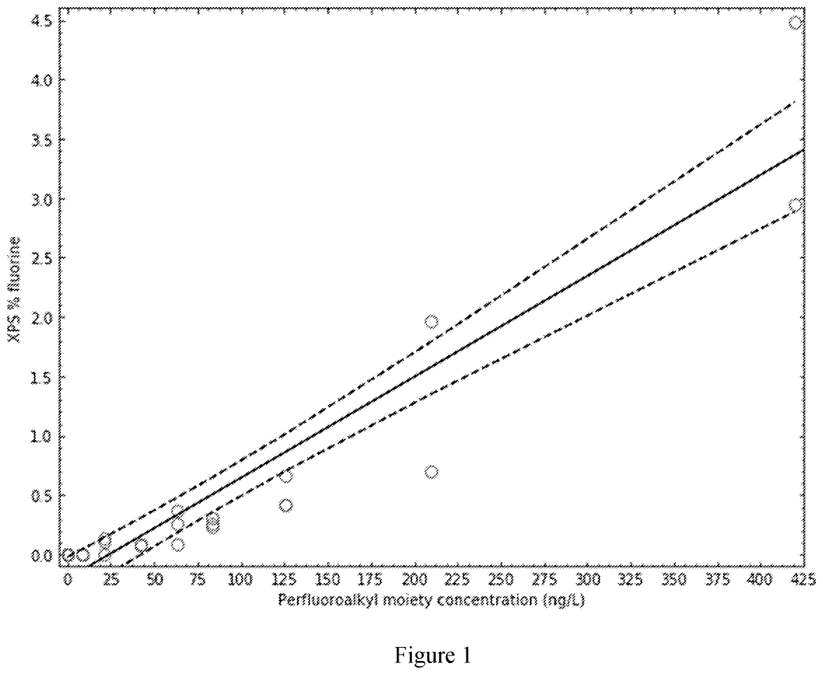

Standards. Standard solutions of PFOS (heptadecafluorooctanesulfonic acid tetraethylammonium salt, 98% purity, Sigma Aldrich) in methanol (Optima grade, Fisher Chemical) were prepared for quantification of PFAS by XPS. 1 μl of each solution was transferred to a 3.175 mm diameter punch of a cellulose acetate membrane filter, (0.45 μm pore size, Sterlitech) by pipet. Standards were used to create a standard curve that relates % F to the perfluorinated mass on a filter (FIG. 1).

Dissolved organic carbon. In order to evaluate potential effects of dissolved organic carbon (DOC) on PFAS signals, groundwater samples were filtered (0.45 μm polypropylene, GH Polypro) and analyzed using high temperature combustion using a Shimadzu TOC-VCPH Total Organic Carbon Analyzer.

XPS. XPS experiments were performed using a Physical Electronics VersaProbe II instrument equipped with a monochromatic Al kα x-ray source (hν=1,486.7 eV) and a concentric hemispherical analyzer. Charge neutralization was performed using both low energy electrons (<5 eV) and argon ions. The binding energy axis was calibrated using sputter cleaned Cu (Cu 2p_{3/2}=932.62 eV, Cu 3p_{3/2}=75.1 eV) and Au foils (Au 4f_{7/2}=83.96 eV). Peaks were charge referenced to CHₓ band in the carbon is spectra at 284.8 eV. Measurements were made at a takeoff angle of 45° with respect to the sample surface plane. This resulted in a typical sampling depth of 3-6 nm (95% of the signal originated from this depth or shallower). Instrumental relative sensitivity factors (RSFs) that account for the x-ray cross-section and inelastic mean free path of the electrons were used to quantify atomic %.

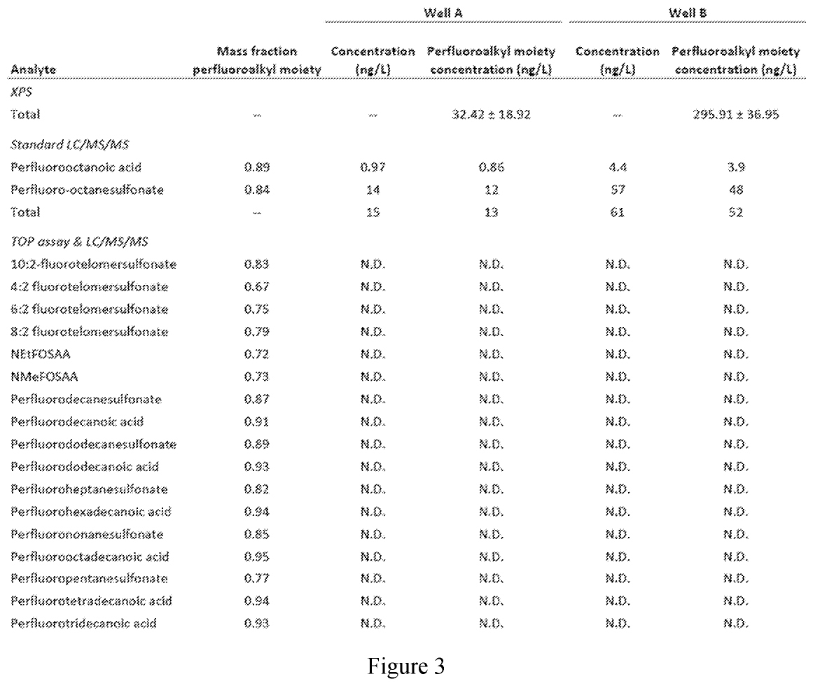

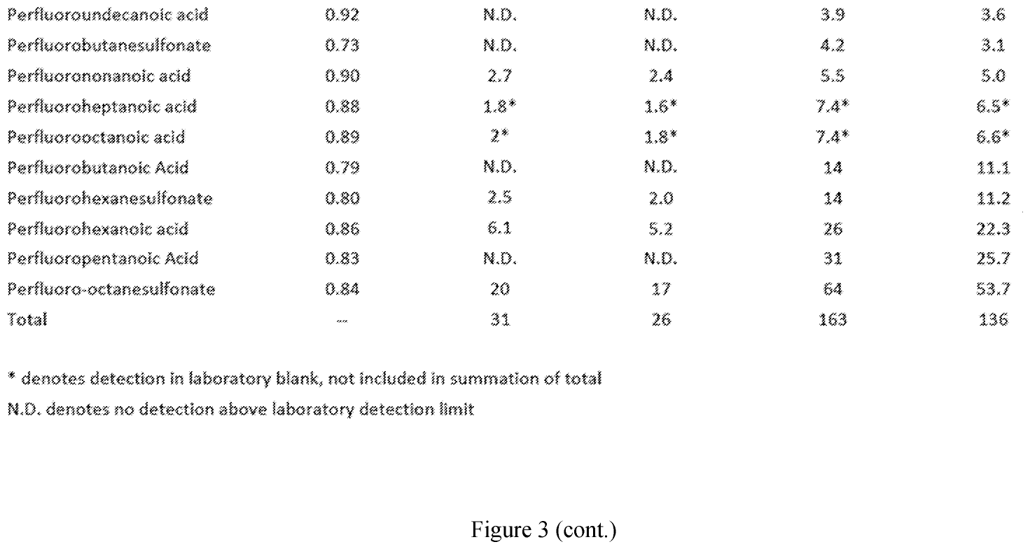

LC/MS/MS. PFASs in groundwater samples and treatment system effluent were measured at Eurofins Lancaster Labs using a modified EPA 537 Version 1.1 method. To measure the most comprehensive suite of PFASs possible, water samples from wells A and B were subjected to a hydroxyl radical-based chemical oxidation to convert polyfluorinated precursors to readily detectable PFAS, a step used in the Total Organic Precursor (TOP) assay. The LC/MS/MS method targeted 27 PFASs (FIG. 3). For simplicity, measurements of PFAS in the post-oxidation samples are referred to as “TOP” data but note that PFASs was not measured in the samples before oxidization, as is done in the standard TOP assay.

Calculation of perfluoroalkyl moiety concentration and comparison of XPS and LC/MS/MS data. PFOS standards were used to determine the relationship between % F measured via XPS and PFOS concentrations (FIG. 1, Table 1).

TABLE 1
XPS Standard Curve Data.

| Perfluoroalkyl moiety mass (ng) | % Fluorine |
|---|---|
| 0.00 | 0.00 |
| 0.00 | 0.00 |
| 0.00 | 0.00 |
| 0.00 | 0.00 |
| 0.00 | 0.00 |
| 0.00 | 0.00 |
| 8.40 | 0.00 |
| 8.40 | 0.00 |
| 8.40 | 0.00 |
| 20.99 | 0.11 |
| 20.99 | 0.14 |
| 20.99 | 0.00 |
| 41.98 | 0.07 |
| 41.98 | 0.09 |
| 62.97 | 0.08 |
| 62.97 | 0.37 |
| 62.97 | 0.26 |
| 83.96 | 0.26 |
| 83.96 | 0.24 |
| 83.96 | 0.31 |
| 125.94 | 0.42 |
| 125.94 | 0.42 |
| 125.94 | 0.66 |
| 209.90 | 0.70 |
| 209.90 | 1.97 |
| 419.80 | 2.95 |
| 419.80 | 4.48 |

The resulting standard curve, however, cannot be used to directly calculate total PFASs in water samples that may contain complex mixtures of unknown PFASs with varying head group masses and perfluoroalkyl tail lengths. To use XPS as a screening tool for complex PFAS mixtures, the XPS standard curve was converted to a unit that represents the total PFAS present in a sample: the perfluoroalkyl moiety concentration. To achieve this for PFOS, the perfluoroalkyl moiety mass fraction (0.84) was calculated by subtracting the mass of the sulfonic acid moiety (80.97 Da) from the PFOS molecule (499.94 Da). The PFOS standard concentrations were then multiplied by 0.84 to generate a perfluoroalkyl moiety standard curve that can be used to estimate the concentration of total perfluoroalkyl moieties in groundwater samples.

For direct comparison of XPS and LC/MS/MS results, LC/MS/MS-derived concentrations must also be converted to perfluoroalkyl moiety concentrations. To do this, the perfluoroalkyl moiety mass fraction for each PFAS detected by LC/MS/MS was determined by subtracting the mass of its head group from its total molecular mass, and multiplied each measured concentration by its respective perfluoroalkyl moiety mass fraction to convert it to perfluoroalkyl moiety concentration (Table 1 and FIG. 3). Finally, the perfluoroalkyl moiety concentrations for the ten PFASs detected by LC/MS/MS were summed for comparison with XPS data.

Results and Discussion

XPS standard curve. XPS consistently detected fluorocarbons in standard samples with PFOS ≥25 ng (21 ng perfluoroalkyl moiety) and detected 0% F (0 ng PFOS) in all blank samples.

There was a positive linear relationship between perfluoroalkyl moiety concentration and % F (FIG. 1). This relationship (Equation 1) was used to determine total perfluoroalkyl moiety concentration in groundwater samples.

Perfluoroalkyl moiety concentration (ng/L)=(% F−0.2044)/(0.0085×L of groundwater) (1)

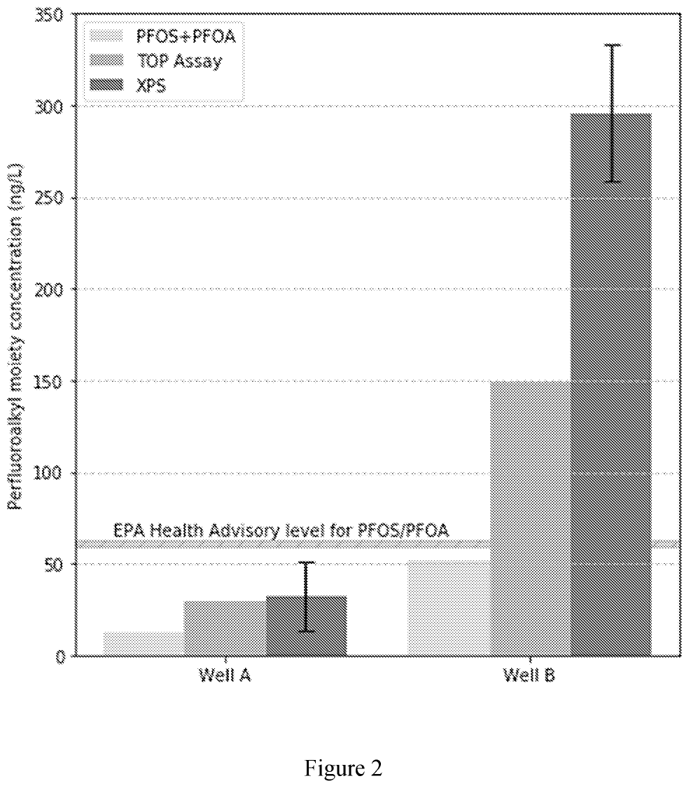

Site specific results. Using XPS, total perfluoroalkyl moiety concentrations were measured in two groundwater samples (Well A and Well B) from the former firefighter training site and compared results with TOP-LC/MS/MS data. Both methods showed that the Well B groundwater contained more total PFAS than Well A groundwater (FIG. 2). Additionally, the XPS method detected no PFAS in a post-GAC treatment effluent sample (FIG. 3). Although no concurrent LC/MS/MS measurement was made, effluent samples collected before and after the aforementioned post-GAC treatment effluent sample had no PFAS detections as measured by LC/MS/MS, consistent with XPS results and indicative of effective removal of PFAS by the GAC.

XPS- and TOP-determined perfluoroalkyl moiety concentrations were higher than the sum of PFOS and PFOA concentrations detected by LC/MS/MS (FIGS. 2 and 3) in both wells, indicating that groundwater below the firefighter training site contains a more extensive suite of PFASs beyond the two most commonly monitored compounds. XPS-determined concentrations were higher than TOP-determined concentrations, likely due to the capability of the XPS method to detect any PFASs captured by SPE. Although TOP greatly expanded the range of PFAS that can be detected in complex environmental mixtures, it has two limitations compared to XPS: some PFAS precursors are not susceptible to oxidation and thus evade detection, and the LC/MS/MS method targets a growing but still limited list of known PFAS.

The pronounced difference between XPS and TOP measurements in Well B may reflect differential transport and degradation of PFASs, since Well A is more distal to the original contaminant source than Well B. It appears plausible that the unknown PFASs revealed by XPS are either less mobile or more short-lived than the majority of the known suite of compounds measured by LC/MS/MS.

This XPS method was developed for low dissolved organic carbon (DOC) groundwater samples. Groundwater samples across the field site have low DOC (0.21-0.36 mg C/liter), as do most drinking water supplies. The C—F signal of PFASs would likely be diluted or masked in higher DOC waters containing significant concentrations of organic compounds that can be captured by SPE. Further method development testing of SPE phases with different molecular selectivity and additional extract cleanup steps could help overcome this limitation, making the method suitable for samples with higher DOC, including wastewater and soil extracts.

A second potential limitation of XPS method is that synthetic fluorocarbons other than PFASs could contribute to the measured % F, falsely inflating PFAS concentrations. Multiple pharmaceuticals and agricultural chemicals in common use are fluorinated, but are likely to only weakly contribute to % F if present because of the limited number of C—F bonds in fluorinated vs. perfluorinated molecules. The pharmaceutical fluoxetine and the insecticide diflubenzuron, for instance, contain only one and two C—F₃ bonds, respectively.

The XPS method reported herein is sensitive enough to screen water at PFAS concentrations well below the EPA Health Advisory level (70 ng/L). The XPS method uses the same SPE prep as EPA Method 537.1 that employs LC/MS/MS. If an adequate volume of water is extracted (˜1.25 L), an aliquot can be reserved for detailed PFAS structural identification and quantification LC/MS/MS in cases in which XPS screening indicates the presence of PFASs at a level of concern. Of the methods available to assess PFAS contamination, XPS captures the broadest scope of the growing PFAS landscape, and could serve as a simpler and more comprehensive drinking water screening method than any currently available.

Although the present disclosure has been described with respect to one or more particular embodiments and/or examples, it will be understood that other embodiments and/or examples of the present disclosure may be made without departing from the scope of the present disclosure.

What is claimed is:

1. A method for detecting one or more fluorocarbon(s) in a sample suspected of having one or more fluorocarbon(s), wherein the sample is disposed on at least a portion of a substrate, comprising:
obtaining an XPS spectrum of the sample;
wherein the presence of a fluorine signal as determined by XPS analysis is indicative of the presence of one or more fluorocarbon(s) in the sample or
the absence of a fluorine signal as determined by XPS analysis is indicative of the absence of one or more fluorocarbon(s) in the sample.

2. The method of claim 1, wherein the XPS analysis comprises determining the strength and/or position of the F 1s peak.

3. The method of claim 1, wherein the XPS analysis comprises one or more high-resolution scans.

4. The method of claim 1, wherein the fluorocarbon(s) is/are chosen from fluoroalkyl compound(s) and combinations thereof.

5. The method of claim 1, wherein the substrate comprises a porous polymer membrane.

6. The method of claim 5, wherein the porous polymer membrane is chosen from hydrocarbon polymers, polyethersulfones (PESs), polyesters, cellulose materials, polycarbonates, functionalized analogs thereof, and combinations thereof.

7. The method of claim 1, wherein the sample is an aqueous sample and/or the method further comprising preparing the sample using an aqueous sample.

8. The method of claim 7, wherein the aqueous sample is chosen from groundwater samples, wastewater samples, potable water samples, drinking water samples, and surface water samples.

9. The method of claim 7, wherein the preparing comprises using solid-phase extraction (SPE).

10. The method of claim 9, wherein the using solid-phase extraction comprises contacting the aqueous sample with an SPE cartridge comprising a polymeric adsorption media, wherein, if fluorocarbon(s) is/are present in the aqueous sample, at least a portion of the fluorocarbon(s) are disposed on the SPE cartridge polymeric adsorption media,
eluting at least a portion of the fluorocarbon(s), if present, from the SPE cartridge polymeric adsorption media to form an SPE eluent, and
optionally, concentrating the SPE eluent to form a concentrated eluent.

11. The method of claim 10, wherein the eluent or the concentrated eluent is contacted with the substrate or a portion thereof and at least a portion of the fluorocarbon(s) is/are disposed on the substrate, which may be used directly in the XPS analysis.

12. The method of claim 1, wherein the XPS analysis comprises irradiating a region of the substrate with incident x-rays, and detecting emitted photoelectrons with a spectrometer.

13. The method of claim 12, wherein the incident x-rays are at an angle of less than 90 degrees and greater than 0 degrees with respect to a surface of the substrate of the region of the substrate.

14. The method of claim 1, further comprising quantifying the amount of fluorine in the sample, wherein the amount of fluorine in the sample correlates to the amount of fluorocarbon(s) in the sample.

15. The method of claim 14, wherein the fluorine quantification is carried out by comparison to one or more standard sample(s).

16. The method of claim 1, wherein the limit of detection of fluorine in the sample is 20 ng or less on a substrate.

17. The method of claim 1, wherein the one or more fluorocarbon(s) is/are per- or polyfluoroalkyl substances PFAS(s).

18. The method of claim 1, wherein the one or more one or more fluorocarbon(s) is/are chosen from perfluorooctanoic acid (PFOA), perfluorooctanesulfonic acid/perfluorooctanesulfonate (PFOS), perfluorobutane sulfonic acid (PFBS), hexafluoropropylene oxide, and combinations thereof.

19. The method of claim 1, wherein XPS analysis comprises determination of the F is peak from 687.0-689.5 eV.
